(19) United States
(12) Patent Application Publication
(10) Pub. No.: US 2004/0234586 A1
(43) Pub. Date: Nov. 25, 2004

(54) Compositions and methods for enhancing nucleic acid transfer into cells

(76) Inventors: Sylvain Meloche, Montreal (CA); Marc Saba-El-Leit, Montreal (CA); Laure Voisin, Montreal (CA)

Correspondence Address: NIXON & VANDERHYE, PC, 1100 N GLEBE ROAD, 8TH FLOOR, ARLINGTON, VA, 22201-4714, US

(21) Appl. No.: 10/480,214

(22) PCT Filed: Jun. 11, 2002
(86) PCT No.: PCT/CA02/00865

Related U.S. Application Data
(60) Provisional application No. 60/296,745, filed on Jun. 11, 2001.

(51) Int. Cl. A61K 9/127

(52) U.S. Cl. 424/450

(57) ABSTRACT

The present invention relates to compositions and methods for enhancing nucleic acid transfer into cells and especially for enhancing nucleic acid transfer into cells for which conventional non-viral transfection methods and compositions are inefficient. More specifically, the present invention is concerned with nucleic acid delivery compositions and methods comprising cationic polymers. The present invention particularly relates to compositions and methods for transfecting cells using a nucleic acid-cationic polymer-lipid mixture. The compositions and methods of the present invention display significantly improved transfection efficiency over other non-viral delivery formulations and methods.

### FIELD OF THE INVENTION

The present invention relates to compositions for enhancing gene transfer into cells and methods using same. More specifically, the present invention relates to compositions comprising cationic polymers vectors and methods using same.

### BACKGROUND OF THE INVENTION

Cell transfection involves delivery of genes to the cell surface followed by an efficient uptake and transport across intracellular barriers to the nucleus of the cells. There are currently five major methods by which this is accomplished, namely: (1) calcium phosphate precipitation; (2) DEAE-dextran complexes; (3) electroporation; (4)-reconstituted viruses or virosomes; and (5) cationic lipid complexes. Transfection is generally optimal when delivery particles are positively charged since anionic cell-surface proteoglycans of adherent cells mediate nonspecific binding and are responsible for their spontaneous internalization (Behr et al., 1989; Mislick and Baldeschwieler, 1996).

Polyethylenimine (PEI), a branched cationic polymer, has been demonstrated to be a highly efficient agent for delivering oligonucleotides and plasmids to cells in vitro (Boussif et al., 1995). Every third atom is an amino nitrogen that can be protonated thus acting as a proton sponge. This high cationic charge-density allows PEI to interact electrostatically with DNA thereby neutralizing the negative charge of DNA, condensing its structure, and thus protecting the DNA from nuclease degradation. The PEI-DNA complexes form compact toroidal structures (Dunlap et al., 1997; Tang and Szoka, 1997; Wagner et al., 1991) called lipoplexes (Felgner et al., 1997). PEI has been shown to promote delivery from the cytoplasm to the nucleus and facilitate transgene expression in the nucleus more efficiently than cationic lipids (Pollard et al., 1998). In cells, PEI's high pH-buffering ability is considered to protect DNA from degradation in the endosome by inducing osmotic swelling of the endosome which results in vesicle lysis and allows the vector-DNA complex to be released (Behr, 1996; Boussif et al., 1995).

The path taken by PEI-DNA complexes used for transfection has been described (Godbey et al., 1999). The use of fluorescent probes to label PEI and DNA has shown that complexes attach to the cell surface and migrate into clumps that are endocytosed. These vesicles increase in number and size and eventually lyse. The complexes are then liberated and undergo nuclear localization. Both PEI associated to DNA or PEI alone are found in the nucleus in the form of ordered structures following transfection.

The size of DNA complexes is known to strongly correlate with relative transfection efficiency. The charge ratio of the polycation to DNA, ionic strength of solution, DNA concentration, or serum content of culture medium are all parameters that influence the size of DNA-PEI particles (Ogris et al., 1998; Tang and Szoka, 1997).

PEI can promote effective gene delivery in a variety of cells: 3T3, HepG2, COS-7, HeLa (Boussif et al., 1996), pancreatic epithelioid CFPAC-1 and lung carcinoma (Pollard et al., 1998). PEI has also been shown to improve transfection efficiency when combined to adenovirus (Baker et al., 1997; Meunier-Durmort et al., 1997) and liposomes (Bandyopadhyay et al., 1998). In vivo, PEI has been used in mouse central nervous system (Goula et al., 1998) and mature mouse brain (Abdallah et al., 1996) and was shown to be promising as an intravenous delivery system (Goula et al., 1998).

Cationic lipid formulations have become popular gene delivery reagents as alternatives to viral delivery vectors. However, it is estimated that only 1 out of 10⁴ plasmid molecules presented to the cell by cationic lipids, reaches the nucleus and is expressed. In addition, many cell types such as fibroblasts and vascular smooth muscle cells (VSMC) have been reported to be particularly difficult to transfect with known lipid formulations. Until now, the focus has been on the synthesis of new cationic lipids and search of new lipidic formulations and these studies have been based on structure-function relationships and ability to transfect cells in vitro.

U.S. Pat. No. 5,945,400 describes a composition for transfecting a nucleic acid, comprising a transfection agent (i.e. a cationic polymer or lipofectant) and a nucleic acid condensation-promoting agent (i.e. a peptide derived from histone, nucleoline or proteonine). In accordance with U.S. Pat. No. 5,945,400, the condensation compound is taught to enable a considerable reduction of the transfection agent. In one embodiment, in addition to the compound involved in condensation of the nucleic acid, the transfection agent is PEI. In another embodiment, the transfection agent is a lipofectant. Thus, while U.S. Pat. No. 5,945,400 teaches compositions comprising a peptide and a cationic polymer such as PEI, or a peptide and a lipofectant, it does not teach nucleic acid transfecting compositions lacking such peptides. It also does not teach such peptide-minus composition further comprising PEI and lipids.

U.S. Pat. No. 5,981,501 teaches a nucleic acid delivery formulation comprising a double layer of lipids. The formulation optionally comprises PEI. The preparation of this formulation depends on a rather complex process which involves multiple steps: 1) a mixing of a nucleic acid with cationic lipids in a detergent; 2) adding non cationic lipids; 3) removing the detergent so as to form double layered micelles.

Of note, delivery vectors and methods using same of the prior art are thought to be inhibited by the presence of serum in the cell growth media. As a consequence, the serum present in the media has to be removed therefrom prior to transfection, to ensure that no inhibitory amount of serum remains in the media surrounding the cells to be transfected. Obviously, this precludes the use of such approaches for several cell types which are highly sensitive to serum deprivation and die by apoptosis. This also precludes any use of the procedure in intact animals.

Furthermore, non-viral transfection compositions of the prior art are unable to achieve the transfection efficiency of the viral transfection methods. Also, methods using reconstituted viruses or virosomes are unable to transfect non-dividing cells. Of note, there has yet to be provided non-viral-based compositions and methods which enable an efficient transfection of primary cells.

There thus remains a need to provide such non-viral-based compositions and methods. There also remains a need to provide methods and compositions which enable an efficient transfection of non-dividing cells. In addition, there remains a need to be provided with a simple method for preparing high efficiency nucleic acid delivery compositions. There also remains a need to provide new nucleic acid delivery compositions whose transfection efficiency is not inhibited by the presence of serum in the growth media of the cells to be transfected and of new methods using same. Also, there remains a need for new non-viral nucleic acid delivery vectors.

There is more particularly a need for efficient, non-toxic and non-viral delivery vectors for in vivo gene delivery.

The present invention seeks to meet these and other needs.

The present description refers to a number of documents, the content of which is herein incorporated by reference in their entirety.

### SUMMARY OF THE INVENTION

The invention relates to nucleic acid delivery compositions comprising at least one lipid and preferably a cationic polymer, the composition enabling an efficient transfection in the presence of serum in the cell culture media. The invention also relates to methods of transfecting cells using the nucleic acid delivery compositions of the present invention.

More particularly, the invention concerns cationic polymer-lipid nucleic acid compositions for transfecting cells including cells in vivo or in culture, cells in suspension or adherent cells using cationic polymer-lipid nucleic acid delivery vectors of the present invention.

The present invention further relates to methods using the cationic polymer-lipid-nucleic acid compositions for transfecting cells.

The Applicant was the first to provide a synergistic combination of cationic polymer and lipids for transfecting cells. In addition, the applicant was the first to show that a combination of cationic polymer and lipids could enable a significant improvement in transfection efficiencies. Moreover, the Applicant was the first to demonstrate that the combination of cationic polymer and lipids is a synergistic combination.

The Applicant provides a method for transfecting cell that is both efficient and simple. The methods of the present invention simply require that all reagents according to the present invention be mixed together without requiring any particular conditions or sequence of addition.

Also, the methods of the present invention require a smaller amount of lipid than certain methods of transfection of the prior art and may therefore be less costly.

Before the present invention, low transfection generating cells could not be transfected efficiently with non-viral vectors. Also, prior to the present invention, most transfection methods were not very efficient when serum was present in the media. Thus the composition of the present invention overcome such drawbacks of the prior art by enabling a high transfection efficiency of cells in the presence of serum and of low transfection generating cells.

In addition, the present invention enables a relatively high efficiency of transfection of primary and/or non-immortalized and/or non-dividing cells in the absence of a viral delivery system.

In accordance with the present invention, there is therefore provided a composition for transfecting cells comprising a nucleic acid-cationic polymer-lipid mixture.

In accordance with the present invention, there is also provided a composition for transfecting cells comprising: polyethyleneimine; a lipid selected from the group consisting of FuGENE 6™, Effectene™, Lipofectamine™, Lipofectine™, 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), and 1,2-Diphytanoyl-sn-Glycero-3-Phosphoethanolamine (DPPE), a natural lipid preparation, a phospholipid and any combination thereof; and a nucleic acid selected from the group consisting of synthetic, natural or modified DNA, RNA and DNA-RNA hybrids. Of course, a mixture of nucleic acids could also be used.

In accordance with another aspect of the present invention, there is provided a use of a composition comprising: a cationic polymer; a lipid selected from the group consisting of FuGENE 6™, Effectene™, Lipofectamine™ and Lipofectine™, 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), and 1,2-Diphytanoyl-sn-Glycero-3-Phosphoethanolamine (DPPE), a natural lipid preparation, a phospholipid and any combination thereof; and a nucleic acid, for transfecting cells in the presence of serum, for transfecting cells such as low transfection generating cells, non-immortalized, non-dividing cells and/or primary cells.

The present invention also relates to a kit for transfecting cells in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the sample (cells to be transfected), a container which contains the nucleic acid, one or more containers which contain one or more cationic polymer and one or more containers which contain one or more lipids.

As taught hereinbelow, in the compositions and methods of the instant invention, the N/P ratio may vary between about 1 and about 50 and is preferably between about 2.5 to 20 and more preferably between about 5 to 10.

The quantity of nucleic acid used in the delivery composition may vary between about 0.10 μg and about 10 μg per 50,000 cells, and is preferably between about 0.25 μg to 5.0 μg per 50,000 cells, and more preferably between about 0.50 μg to to 2.5 μg per 50,000 cells.

When FuGENE 6™ is used, the lipid/nucleic acid ratio may vary between about 0.25 μl to about 10 μl per pg of nucleic acid acid, and is preferably between about 0.50 μl to 5.0 μl per pg of nucleic acid acid, and more preferably between about 1.0 μl to 3.0 μl per pg of nucleic acid acid.

When synthetic lipids are used at a concentration of 2 mg/ml, the lipid/nucleic acid acid ratio may vary between about 0.10 μl to about 15 μl per pg of nucleic acid acid, and is preferably between about 0.25 μl to 7.5 μl per pg of nucleic acid acid, and more preferably between about 1.0 μl to 6.0 μl per pg of nucleic acid acid.

It was observed that the transfection efficiency increases within these ranges when the N/P ratio, the nucleic acid quantity and the lipid/nucleic acid ratio increases.

In order to provide a clear and consistent understanding of terms used in the present description, a number of definitions are provided hereinbelow.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, N.Y.).

Examples of cationic polymers may be found for instance in U.S. Pat. No. 5,981,501. Non-limiting examples include polyethyleneimine, poly-L-ornithine, poly-L-arginine, poly-L-lysine, poly-D-lysine, polyallylamine, polypropyleneimine.

Numerous lipids are suitable for compositions of the present invention. Non-limiting examples of lipids include phospholipids, lipids having phosphocholine or phosphoethanolamine as headgroup, 16-18 carbon chains containing lipids including those having phosphocholine or phosphoethanolamine as headgroup and in particular, 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), and 1,2-Diphytanoyl-sn-Glycero-3-Phosphoethanolamine (DPPE), FuGENE 6™, Effectene™, Lipofectamine™ and Lipofectine™.

As used herein, “nucleic acid molecule”, refers to a polymer of natural, synthetic or modified (e.g. phosphorothioates) nucleotides or a mixture thereof. Non-limiting examples thereof include DNA (e.g. genomic DNA, cDNA), RNA (e.g. mRNA, ribozymes, catalytic RNAs, interference RNA (RNAi) and small interference RNA (siRNA) ), DNA-RNA hybrids, etc. One skilled in the art would know how to adapt the methods and compositions of the present invention for transfecting cells with a chosen nucleic acid. The nucleic acid molecule can be obtained by cloning techniques or synthesized. DNA can be double-stranded or single-stranded (coding strand or non-coding strand [antisense]). The nucleic acid for practicing the present invention may be obtained according to well-known methods.

The term “DNA” molecule or sequence (as well as sometimes the term “oligonucleotide”) refers to a molecule comprised generally of the deoxyribonucleotides adenine (A), guanine (G), thymine (T) and/or cytosine (C) and or derivatives thereof, usually in a double-stranded form (modified or rare bases are well known in the art). Such molecules may comprise or include a “regulatory element” as the term is defined herein.

The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. Of course, single stranded DNA molecules and oligonucleotides can be used in accordance with the present invention

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well-known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As used herein, the term “gene” is well-known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into any one of numerous established kit formats which are well-known in the art.

The term “vector” is commonly known in the art and defines a plasmid DNA, phage DNA, viral DNA and the like, which can serve as a DNA vehicle into which DNA can be cloned. Numerous types of vectors exist and are well-known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences (or regulatory elements) is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

The level of gene expression of the reporter gene (e.g. the level of luciferase, or β-galactosidase produced) within the transfected cells according to the present invention can be compared to that of the reporter gene in transfected cells according to methods and with compositions of the prior art or other controls. The difference between the levels of gene expression obtained with the methods and compositions of the present invention and those obtained with methods and compositions of the prior art indicates whether the methods and compositions of the instant invention improve transfection efficiency.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

A host cell or an indicator cell has been “transfected” by exogenous or heterologous nucleic acid (e.g. a nucleic acid construct) when such nucleic acid has been introduced inside the cell. When the transfecting nucleic acid is DNA, it may or may not be integrated (covalently linked) into chromosomal DNA making up the genome of the cell. In prokaryotes, yeast, and mammalian cells for example, the transfecting DNA may be maintained on an episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well-known in the art (Sambrook et al., 1989, supra; Ausubel et al., 1994 supra).

As used herein, the designation “low transfection generating cells” refer to cells (e.g. non-dividing or [dividing cells, cells in suspension, adherent cells, primary cells, non immortalized, etc.) that are difficult to transfect with the compositions and methods of the prior art. Non-limiting examples of such low transfection generating cells include primary cells such as: astrocytes, cardiomyocytes, chondrocytes, chromaffin cells, aortic endothelial cells, cardiac endothelial cells, coronary artery endothelial cells, lung vein epithelial cells, mammary epithelial cells, prostate epithelial cells, tracheal epithelial cells, umbilical vein endothelial cells, fibroblasts, embryonic fibroblasts, skin fibroblasts, hepatocytes, aortic vascular smooth muscle cells, coronary artery smooth muscle cells, jejunum smooth muscle cells, umbilical vein smooth muscle cells, keratinocytes, embryonic muscle cells, skeletal muscle myoblasts, neurons, oligodendrocytes, retinal pigment epithelial cells, sertoli cells, embryonic stem cells, thyroid cells, uterine stromal cells; and established cell lines such as: HEK-293 (human epithelial kidney), 3T3 (mouse embryo fibroblasts), B-cells, BHK (hamster kidney fibroblast), Caco-2 (human epithelial), CHO (hamster epithelial ovary), COS (monkey kidney fibroblast), HeLa (human epithelial cervix carcinoma), Hep G2 (human epithelial hepatoblastoma), Huh-7 (human hepatoma), Jurkat (human lymphoma), MCF-7 (human epithelial breast cancer), MDCK (dog kidney epithelial), MEF (mouse embryo fibroblast), Neuro-2A (mouse neuroblastoma), P19 (mouse embryonic teratocarcinoma), PC-12 (rat pheochromocytoma), Phoenix (retroviral packaging cell line), Rat-1 (rat fibroblast), SF9 (fall armyworm epithelial ovary), VSMC (rabbit aortic vascular smooth muscle), and U-937 (human monocyte).

The term “primary cells” is well-known in the art. For certainty, a definition thereof is cells which have never been passed in culture. Such cells are often refractory to transfection. Other cells that are refractory to transfection include cells which have a finite number of divisions in culture. The present invention provides the significant advantage that it enables a significant transfection efficiency of low transfection generating cells, of non-immortalized, non-transformed or normal cells, whether such cells are dividing or non-dividing.

Typical transfection yields with rodent vascular smooth muscle cells (VSMCs), an example of cells which are refractory to transfection or which are considered to be an example of low transfection generating cells, have been of less than 1% when using commercial non-viral agents. As shown below, the compositions of the present invention enable transfection efficiencies at least ten fold higher as compared to known formulations.

As used herein, the designation “FuGENE 6™” is meant to refer to the transfection reagent composition marketed under this trade-mark by the company Boehringer Mannheim under the identification no. 1 814 443. It is described as a proprietary blend of lipids (non-liposomal) formulation and other compounds in 80% ethanol. This transfection reagent is an expensive reagent.

While the transfection efficiencies of the compositions and methods of the instant invention are demonstrated with the use of Rat1 fibroblast cells and VSMC cells, neuronal cells and primary mouse fibroblasts (MEFs), four different model cell cultures of “low transfection generating cells”, the present invention should not be so limited. Indeed, the composition and methods of the present invention can be used with a wide variety of types of normal, non-immortalized, transformed, non-dividing and/or primary cells and cell lines, whether of the “low transfection generating cells” type or not. It is therefore understood that these compositions and methods may be used with a wide variety of cell lines.

The results presented herein indicate that the same cationic polymer-nucleic acid lipid formulation can be used to transfect different cell lines that respond differently when transfected with commercial agents. For example, in both Rat1 fibroblasts and VSMCs, the cationic polymer-nucleic-acid lipid formulation increased significantly transfection efficiencies suggesting that this transfection formulation may also increase transfection yield of a wide variety of cell lines. Furthermore, these results suggest that the improved transfection yields are based on properties that are intrinsic to the nucleic acid lipid formulation and that the degree to which the formulation can successfully transfect a cell line is affected by the cell line itself. In any event, the compositions of the present invention are shown to significantly increase the transfection efficiency in a number of different cells. In view of this teaching, of the different compositions which are taught and of the methods taught herein to monitor the transfection efficiency, a person of ordinary skill could adapt the teachings of the present invention to a particular cell or cell line without undue experimentation.

It will also be clear to the person of ordinary skill that although the efficiency of the transfection compositions and methods of the instant invention have been demonstrated with the use of a representative reporter gene, commonly known in the art, namely pCMV-LacZ DNA, to facilitate visualization of the results, the instant invention may be used for the transfection of any type of nucleic acid molecule with the necessary adaptations (e.g. RNA, DNA, hybrids thereof) and other reporters to monitor the efficiency.

The person of ordinary skill in the art can choose the optimal conditions for formulating cationic polymer-nucleic acid lipid complexes to ensure proper interaction between the polycation, lipid, and nucleic acid in order to enable maximum nucleic acid transfer efficiency and to suit particular needs.

In view of the increased transfection efficiencies taught herein, the present invention provides the means to lower the amount of nucleic acid to be transfected which can be of significance under certain conditions.

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

The compositions of the present invention (as exemplified herein) can be administered to an animal (including humans). In such situation, the formulation can be adapted for such purposes. Excipients and carriers are known in the art (Remington Pharmaceutical Sciences (1980)).

From the specification and appended claims, if used for a therapy, the formulations of the invention can be introduced into animals or individuals in a number of ways, well known in the art. For example, erythropoietic cells or other cells can be isolated from the afflicted individual or animal, transfected with a formulation according to the invention and reintroduced to the afflicted individual in a number of ways, including intravenous injection. Alternatively, formulation can be administered directly to the afflicted individual, for example, by injection in the bone marrow

For administration to humans, the prescribing medical professional will ultimately determine the appropriate form and dosage for a given patient, and this can be expected to vary according to the chosen therapeutic regimen (e.g. DNA construct, protein, cells), the response and condition of the patient as well as the severity of the disease.

Composition within the scope of the present invention should contain the active agent in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

Although the present invention has been illustrated with references to embodiments where the polycationic polymer is PEI, other polycationic polymers are suitable and one skilled in the art will know which other polymers can be used in accordance with the methods and compositions of the present invention.

### BRIEF DESCRIPTION OF THE DRAWINGS

Having thus generally described the invention, reference will now be made to the accompanying drawings, showing by way of illustration a preferred embodiment thereof, and in which:

FIG. 1 shows DNA delivery in Rat1 fibroblast cells using PEI in panel A and FuGENE 6™ in panel B;

FIG. 2 shows the transfection efficiency of PEI, FuGENE 6™ or PEI-FuGENE 6™ in Rat1 fibroblast cells;

FIG. 3 shows the X-Gal staining of Rat1 fibroblast cells transfected with PEI, FuGENE 6™ or PEI-FuGENE 6™;

FIG. 4 shows the comparison of transfection efficiency of PEI-FuGENE 6™ to commercial transfection agents in Rat1 fibroblast cells;

FIG. 5 shows the transfection efficiency of PEI, FuGENE 6™ and PEI-FuGENE 6™ in rat aortic vascular smooth muscle cells (VSMCs);

FIG. 6 shows the transfection efficiency of PEI-FuGENE 6™compared to PEI alone in rat aortic vascular smooth muscle cells (VSMCs);

FIG. 7 shows the comparison of transfection efficiency of PEI-FuGENE 6™ with commercial agents in VSMCs;

FIG. 8 shows the influence of formulation media and serum content on transfection efficiency;

FIG. 9 shows the optimization of transfection efficiency using different formulations of PEI-FuGENE 6™ on Rat1 fibroblast cells (left panel) and VSMCs (right panel).

FIG. 10 shows the effect of cell number and amount of agent on transfection efficiency in Rat1 fibroblast cells (panel A) and in VSMCs (panel B);

FIG. 11 shows the transfection efficiency of natural lipid preparations combined with PEI;

FIG. 12 shows the transfection efficiency of synthetic lipid preparations combined with PEI;

FIG. 13 shows the transfection efficiency of mixtures of synthetic lipids in combination with PEI;

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

The present invention relates to compositions for enhancing nucleic acid transfer into cells and methods using same. Although these methods and compositions may be used to transfect any type of cell, they are particularly useful to the transfection of cells for which conventional transfections are inefficient. More specifically, the present invention is concerned with nucleic acid delivery compositions comprising polyethylenimine and methods using same. The present invention particularly relates to compositions for transfecting cells comprising a polyethylenimine-lipid mixture encapsulating nucleic acid and methods using same. The compositions and methods of the present invention display significantly improved transfection efficiency over other non-viral delivery formulations and methods.

The present invention is illustrated in further detail by the following non-limiting examples.

### EXAMPLE 1

### Preparation of Plasmids

Plasmids pCMV-LacZ encoding β-galactosidase (β-gal) were transformed in E. Coli JM109. Plasmid DNA was isolated from overnight cultures of JM109 and purified by affinity chromatography on QIAGEN columns (Qiagen, Chatworth, Calif., USA) according to manufacturer's protocol. The quality of the DNA was determined by UV spectroscopy and agarose gel electrophoresis (1% agarose) with 1 μg/mL ethidium bromide.

### EXAMPLE 2

### Preparation of Polycationic Polymer-Nucleic Acid and Polycationic Polymer Nucleic Acid-Lipid Complexes

PEI 25 kDa was obtained from Aldrich Chemicals (Milwaukee, Wis., USA) as a 50% w/v solution in water. As described by Boussif et at (Boussif et al., 1995), a 10 mM stock solution was made by mixing 9 mg of PEI in 10 ml of deionised water, adjusting the pH to 7.5 with HCl, and passing the solution through a 0.2-micron filter. The filtered stock solution was stored at 4° C. The 10 mM solution was vortexed vigorously before use,.

Transfection complexes were prepared as described by Boussif et al. (Boussif et al., 1995). Briefly, indicated amounts of DNA and PEI were each diluted in 50 μl 0.15 M NaCl, homogenized and incubated at room temperature. After 5-10 minutes, the PEI was added dropwise to the DNA, homogenized and left for 20 min at RT before addition to cells.

Plasmid DNA (1 μg) and PEI (1 nmol DNA phosphate per 10 nmol amine) were each diluted in 50 μl 0.15 M NaCl. Lipid was diluted in 100 μl of growth media without serum and antibiotics. The solutions were immediately homogenized and left at room temperature for 5-10 min. PEI solution was added to the DNA followed by addition of the lipid to the resulting solution. The combined material was vortexed and incubated at room temperature for 20 min before addition to the cells.

### EXAMPLE 3

### Cell Cultures

Rat1 fibroblast cells were maintained in modified Eagle medium (MEM) (Life Technologies) and VSMC cells were maintained in Dulbecco's modified Eagle medium (DMEM) (Life Technologies). Both cell lines were cultured in media containing 10% heat inactivated calf serum (Life Technologies), 1% penicillin-streptomycin, and L-glutamine in 10 cm tissue culture flasks at 37° C. in a humidified 5% CO₂ atmosphere. Cells were grown to 80% confluency, detached with trypsin-EDTA and replated in 2 new flasks 18-24 h before transfection. Two to three hours before transfections, cells were again detached, and seeded at a density of 5×10⁴ cells per well in 24-multiwell tissue culture plates.

### EXAMPLE 4

### Transfection Procedures

At the time of transfection, cells were supplemented with 200-400 μl of fresh complete media containing 10% calf serum and antibiotics unless otherwise specified. Transfection solution was added to each well and the plates were incubated at 37° C. in a humidified atmosphere containing 5% CO₂. After 6 h of transfection, the transfecting solution was removed, cells were washed once with appropriate cell culture medium and then covered with 1 ml of the same medium. Cells were maintained for an additional 36-48 h.

### EXAMPLE 5

### Determination of Transfection Efficiency

Following transfections with pCMV-LacZ DNA (36-48 h later), cells were washed twice with ice-cold 0.1 M phosphate buffered saline (PBS) and lysed with 250 μl lysis buffer (10 mM Tris-HCl pH 7.5, 1 mM EDTA, 100 mM NaCl, 5 mM MgCl₂ and 1% Nonidet P40™ (Boehringer Mannheim)). Lysates were centrifuged 15 000×g at 4° C. for 5 min. The supernatant was collected and its protein concentration was determined using the BCA protein assay kit (PIERCE) using the microwell protocol according to the manufacturer's specifications. β-galactosidase assay was performed as described by Eustice et al. (Eustice et al., 1991) Briefly, β-galactosidase activity was measured in 240 μl assay mixture containing 80 mM sodium phosphate buffer, pH 7.3, 102 mM 2-mercaptoethanol, 9.0 mM MgCl₂ and 8.0 mM ONPG (o-nitrophenyl-β-D-galactopyranoside, SIGMA). Samples, (1-40 μl) of each lysate were assayed in the reaction mixture at 37° C. for 30 min in 96-well plates. Absorbance was measured at 405 nm. E. coli β-galactosidase (Roche) was used to generate a standard curve.

### EXAMPLE 6

### X-Gal Staining

Following transfections with pCMV-LacZ DNA (36-48 h later), cells were washed twice with ice-cold 0.1 M phosphate buffered saline (PBS) and fixed in glutaraldehyde 0.05% in PBS for 5 min. Cells were washed twice with PBS following fixation and 1 ml stain solution (1 mg/ml 5-bromo-4-chloro-3-indolyl β-D-galactopyranoside (X-gal), 5 mM ferriferrocyanide, 1 mM MgCl₂ in PBS) was added.

### EXAMPLE 7

### PEI-Mediated Gene Transfer in Rat1 Fibroblasts

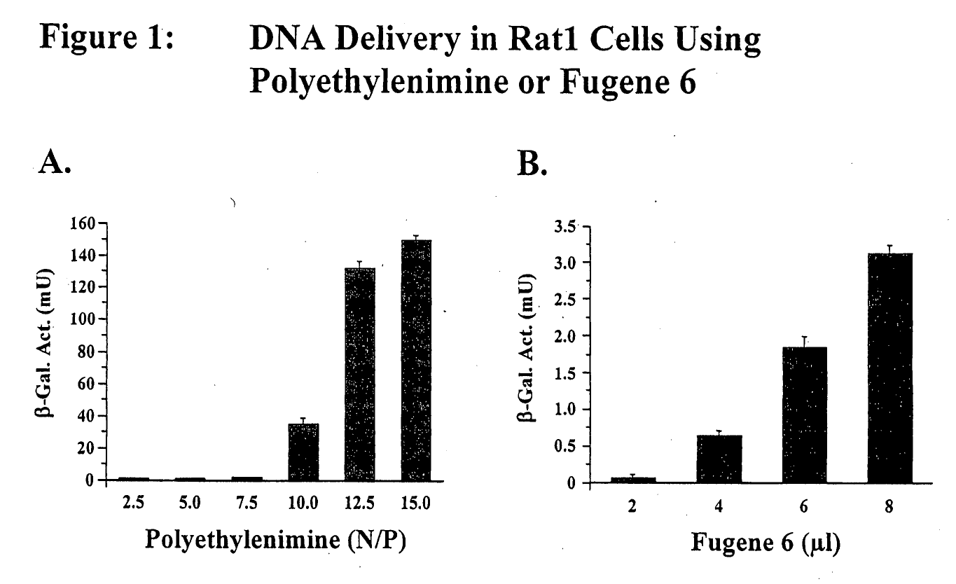

Various quantities of PEI were used to condense pCMV-LacZ plasmid DNA encoding the β-galactosidase (β-gal) enzyme in order to assess the influence of the amount of PEI on gene transfection efficiency. Gene delivery was tested with the resulting complexes by measuring the β-gal activity generated by successful introduction into cells of the pCMV-lacZ plasmid DNA. PEI-nucleic acid complexes were prepared with 2 μg pCMV-LacZ DNA, prepared according to Example 1, and various amounts of PEI and overlaid on Rat1 fibroblast cells (50,000 cells per well in a 24 well plate) according to Example 2. In physiological salt conditions, complexes became positively charged at an N/P ratio >7.5. As is illustrated in FIG. 1, panel A, successful DNA delivery was obtained at PEI nitrogen to DNA phosphate ratios (N/P) of 10 for Rat1 fibroblast cells. Complexes generated at ratios of 10 were shown to have a net positive charge since lower ratios yielded very low gene delivery. The optimal transfection efficiency of PEI-nucleic acid (i.e. DNA) in Rat1 fibroblast cells was obtained at a N/P ratio of 15. At N/P ratios higher than 15, toxicity was observed in cells as demonstrated by lower total protein content (data not shown).

### EXAMPLE 8

### Lipid-Mediated Gene Transfer in Rat1 Fibroblasts

The transfection efficiency of a lipid formulation on the Rat1 fibroblast cells was determined. FuGENE 6™ is a non-viral delivery agent composed of a proprietary blend of lipids and other components (Boehringer Mannheim). Different volumes of FuGENE 6™ were used to transfect 2 μg pCMV-LacZ DNA according to the manufacturer's recommendations. Transfections yielded detectable transgene expression at a FuGENE 6™ to DNA ratio of 2 μl/μg. DNA was combined with different amounts of lipid to detect optimal efficiency in Rat1 fibroblast cells. Lipid formulation at a 4:1 ratio was found to be maximal (FIG. 1B). At lower lipid concentrations, the transfection efficiency was significantly lower. Maximal transfection efficiency for the lipid formulation was discerned to be significantly lower than the maximum observed with PEI transfection according to the present invention, when comparing total β-galactosidase activity (FIG. 1).

### EXAMPLE 9

### Transfection Efficiency of Various PEI-Nucleic Acid-Commercial Transfection Agents Complexes in Rat1 Fibroblast Cells

In order to achieve higher transfection efficiency, PEI was combined with various types of transfection agents including other commercial lipids such as FuGENE 6™, Effectene™; Lipofectamine™ and Lipofectine™ and the activated dendrimer Superfect™ (data not shown) and tested on Rat1 cells and VSMCs (rat aortic smooth muscle cells). Synergetic results observed with both cell lines, with some of these lipids while the increase was not quantified, formal stimulation of transfection efficiency was increased. Hence, the commercial lipids Lipofectamine™, Lipofectin™, and Effectene™ were tested in combination with PEI. Although these combinations displayed increased efficiency relative to PEI alone or a lipid alone, none were as efficient as the PEI-FuGENE 6™ combination in increasing transfection efficiency. These results are based on β-galactosidase activity as observed by X-Gal coloration. Superfect™ reagent in combination with PEI did not increase transfection efficiency (data not shown).

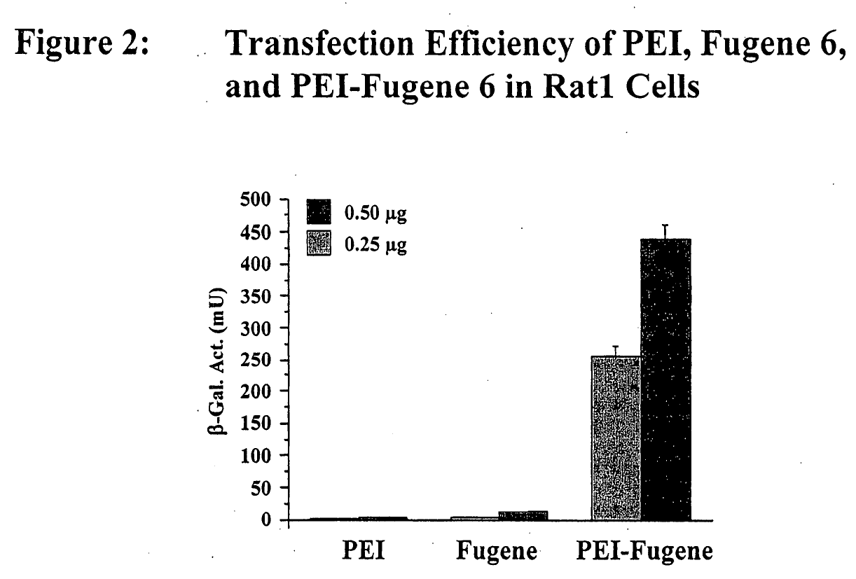

PEI, FuGENE 6™, and a combination of PEI-FuGENE 6™ were used to transfect 0.25 and 0.50 μg of pCMV-LacZ DNA as described in Example 4. Cells were lysed 24-48 h post transfection and the lysate was used to measure protein content and β-galactosidase activity. This resulted in transfection efficiencies remarkably higher than additive efficiency of each component alone. Rat1 fibroblast cells were transfected with 0.5 or 0.25 μg of DNA complexed with PEI at an N/P ratio of 10 and combined with FuGENE 6™ to form a gene delivery complex. Table 1 presents results of these transfection in terms of optical density per mg (O.D./mg). These results are also presented graphically in FIG. 2.

TABLE 1

| Agent | Average O.D./Mg | Sem O.D./Mg |
|---|---|---|
| PEI 0.25 μg DNA | 0.023 | 0.018 |
| PEI 0.50 μg DNA | 0.420 | 0.054 |
| FuGene 0.25 μg DNA | 2.193 | 0.085 |
| FuGene 0.50 μg DNA | 9.590 | 0.085 |
| PEI-FuGene 0.25 μg DNA | 254.333 | 11.675 |
| PEI-FuGene 0.50 μg DNA | 436.800 | 13.472 |

Of the tested formulations, the optimal combination Qf PEI-nucleic acid-lipid complex (0.5 μg) resulted in a transfection efficiency 12-fold higher than that of the highest efficiency observed with PEI-nucleic acid complex using 2.0 μg of DNA at an N/P ratio of 10 (FIG. 1A). Very low β-gal expression was observed with PEI-plasmid complexes when the DNA dose was reduced to 0.25 μg or 0.50 μg per 5×10⁴ cells. In fact, the PEI-nucleic acid-lipid combination has an efficiency of over 1000-fold when compared to the PEI-nucleic acid alone when using similar DNA quantities per well (FIG. 2). Furthermore, a dose-dependent increase in gene delivery was observed when increasing amounts of the PEI-nucleic acid-lipid formulation were used to transfect Rat1 fibroblast cells (FIG. 2).

Interestingly, cells transfected with the lipid formulation and stained with X-gal solution, resulted in at least 10-fold more stained cells as compared to different PEI formulations (data not shown). This suggests that transfections using PEI alone result in less overall percentage of cells expressing reporter gene but significantly higher levels of expression in those cells that are transfected with the polycation. These results are in agreement with earlier work showing that PEI is a more efficient nuclear transport vector (Pollard et al., 1998).

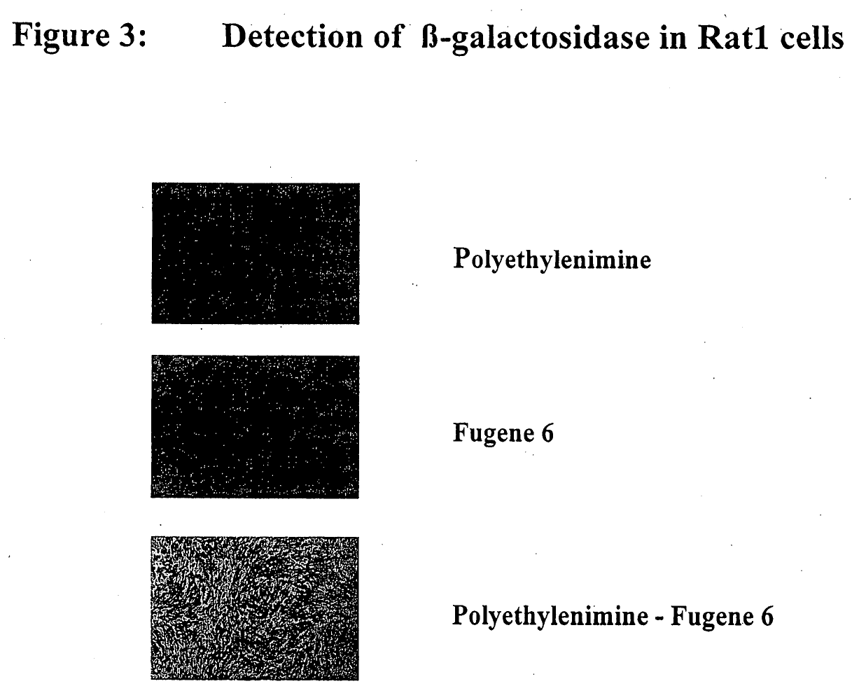

PEI, FuGENE 6™, or a combination of PEI-FuGENE 6™ were used to transfect 0.50 μg of pCMV-LacZ DNA as described in Example 4. Cells were rinsed in PBS and fixed with glutaraldehyde before addition of X-Gal staining solution. Typical results are visualized by detection of β-gal present in cells as detected by X-gal staining (FIG. 3). These results suggest that the use of PEI in combination with FuGENE 6™ dramatically increase transfection efficiency. The transfections shown in FIG. 2 were carried out using a N/P ratio of 10 and a lipid to DNA ratio of 3 which are suboptimal as shown in FIG. 1, suggesting that the combination of the two agents works in a synergistic fashion.

One possible explanation for the significantly higher efficiency using a PEI-nucleic acid-lipid combination of the present invention is that the complexes formed contain both PEI and lipid and that both agents contribute to the gene delivery process. The PEI may contribute by efficiently compacting the DNA thereby protecting it from degradation inside and outside the cells. The lipid might allow a higher percentage of complex uptake by the cells. Within the cells, the presence of PEI in the complex could protect the nucleic acid from degradation in the endosome and facilitate escape therefrom. Once released, the PEI would ensure a more efficient transport towards the nucleus and release inside the nucleus, resulting in higher levels of gene expression.

### EXAMPLE 10

### Transfection Efficiency of PEI-Nucleic Acid-Lipid Complexes in Rat1 Fibroblast Cells as Compared to Commercial Transfection Agents

Rat1 fibroblast cells were transfected with a variety of commercially available transfection agents and a conventional method consisting of calcium phosphate in order to compare their transfection efficiencies to PEI-nucleic acid-lipid transfection compositions of the instant invention. PEI-FuGENE 6™ were used to transfect 0.50 μg of pCMV-LacZ DNA as described in Example 4. Transfections using commercial agents were conducted according to the manufacturer's recommendations.

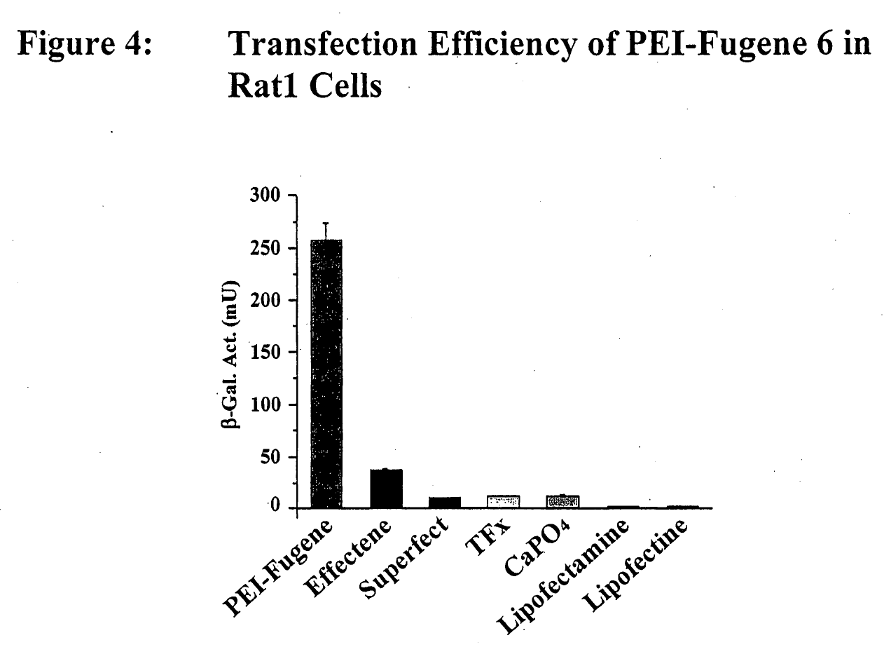

As seen in FIG. 4, the PEI-nucleic acid-lipid complex is significantly more efficient than all the other agents tested. The PEI-nucleic acid-lipid complex generated an efficiency 25 fold higher than that observed with calcium phosphate. The PEI-nucleic acid-lipid complex method was found to yield a 7.5-fold higher transfection efficiency when compared to transfections done with Effectene™ (Qiagen) which yielded the highest efficiency amongst the commercial agents tested.

### EXAMPLE 11

### Transfection Efficiency of PEI-Nucleic Acid-Lipid Complexes in Rat Aortic Vascular Smooth Muscle Cells

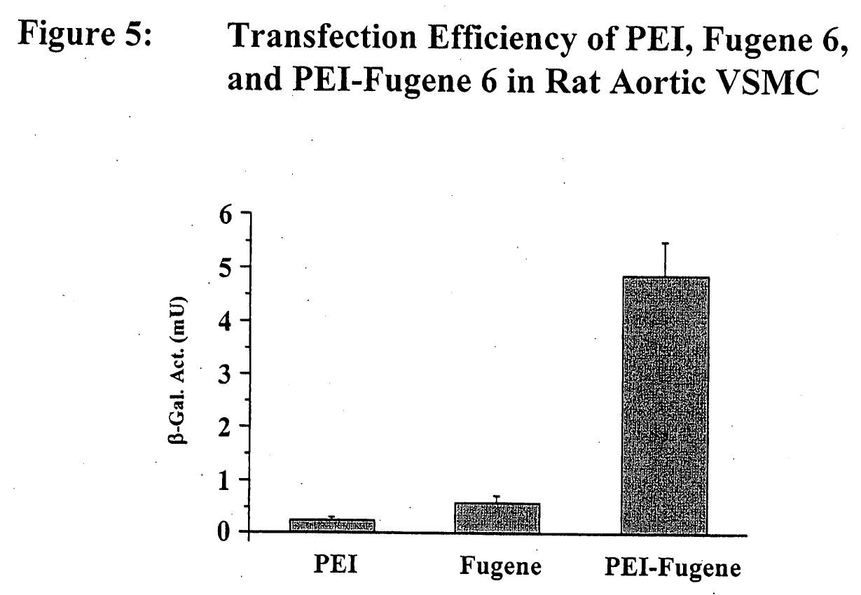

Rat aortic vascular smooth muscle cells (VSMCs) are known to be very refractory to transfection. Indeed, VSMCs generate very low transfection efficiencies using a wide range of transfection agents. Typical transfection yields are less than 1% when using commercial agents. PEI-nucleic acid-lipid compositions of the present invention were thus tested to assay their transfection efficiency using this low transfection generating cell line and determine how it compared to those obtained with known transfection agents. PEI, FuGENE 6™, and a combination of PEI-FuGENE 6™ were used to transfect 0.25 μg of pCMV-LacZ DNA as described in Example 4. Cells were lysed 24-48 h following transfection and lysate was used to measure protein content and β-galactosidase activity. Cells were transfected with 0.25 μg of DNA complexed to PEI (N/P=10) and lipid ratio of 3:1. The PEI-nucleic acid-lipid complex generated a β-gal activity 24-fold higher than the PEI-nucleic acid complex alone (FIG. 5).

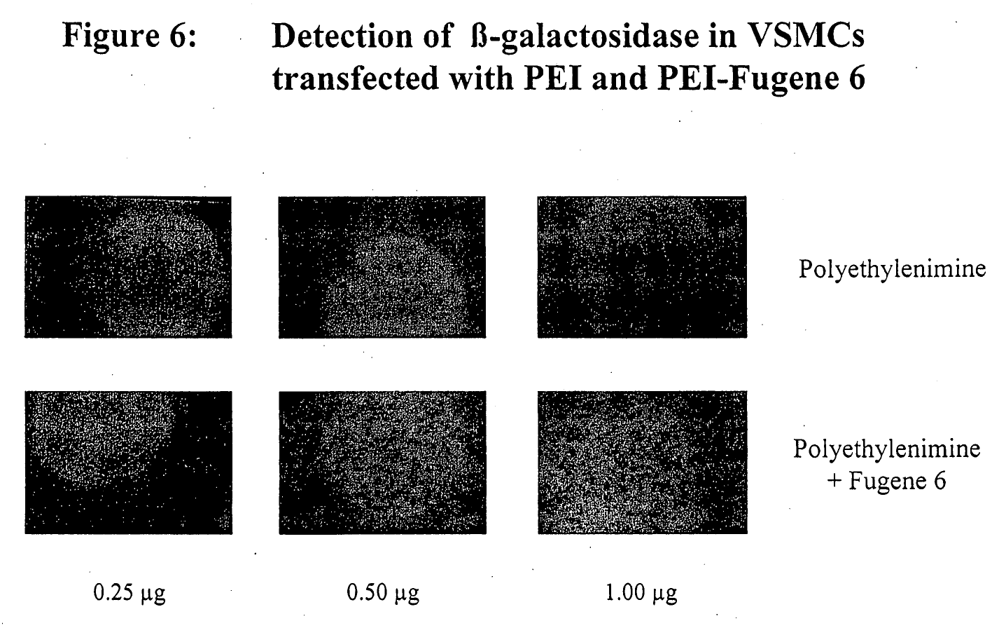

Different amounts of pCMV-LacZ DNA (0.25, 0.50, and 1.00 μg) were combined with appropriate ratios of PEI and lipid as described in Example 4, and the resulting compositions were used to transfect VSMCs. A dose-dependent increase in gene delivery was observed as measured by β-gal activity without any toxic effect over the doses used (not shown). Results from this experiment were visualized by β-gal staining (FIG. 6). Transfection efficiency is compared to that of PEI-nucleic acidalone using corresponding amounts of DNA for the transfection procedure.

### EXAMPLE 12

### Comparison of Transfection Efficiencies of PEI-Nucleic Acid-Lipid Complexes in VSMCs with Other Known Transfection Agents

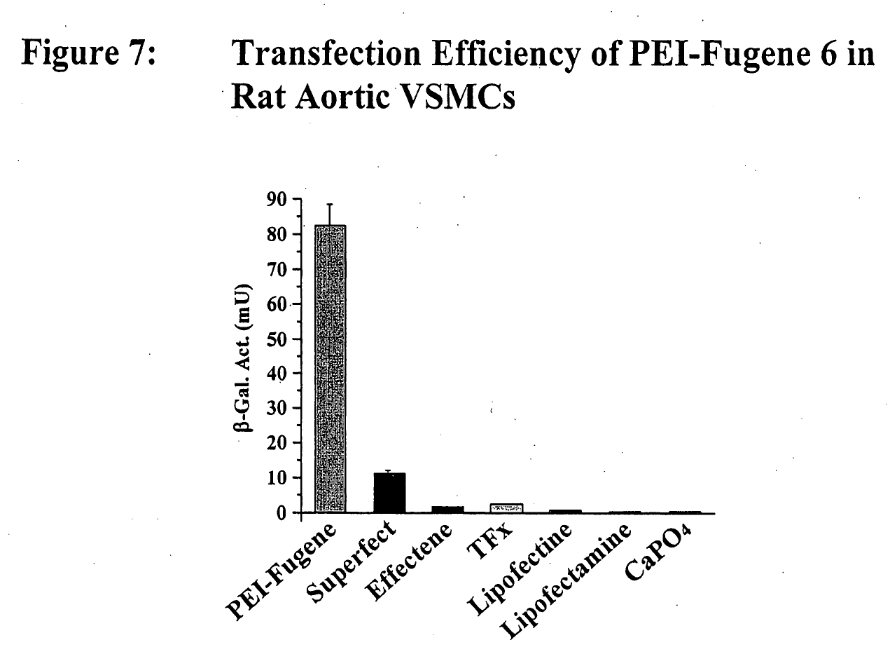

PEI-FuGENE 6™ compositions was used to transfect 1.0 μg of pCMV-LacZ DNA as described in Example 4. Transfections using commercial agents were conducted according to the manufacturer's recommendations. When the transfection efficiency of PEI-nucleic acid-lipid complexes was compared to other commercial transfection agents, the PEI-nucleic acid-FuGENE 6™ complex showed an 8-fold increase in transfection of VSMCs, over the commercial agent achieving the best transfection in VSMCs, namely Superfect™ (FIG. 7).

### EXAMPLE 14

### Parameters Influencing Transfection Efficiency

The state of condensation of plasmid DNA changes with the composition of the formulation medium. Ionic strength of formulating media influences the formation of gene delivery complexes as shown by differences in transfection efficiencies. The influence of PEI-nucleic acid-lipid complexes in different nucleic acid condensation buffers on transfection efficiency was therefore investigated and exemplified with PEI-DNA. PEI-FuGENE 6™ compositions were used to complex 0.25 μg of pCMV-LacZ DNA in the presence of 150 mM NaCl or MEM media (cell culture medium). Transfections of Rat1 cells were conducted in the presence or absence of serum.

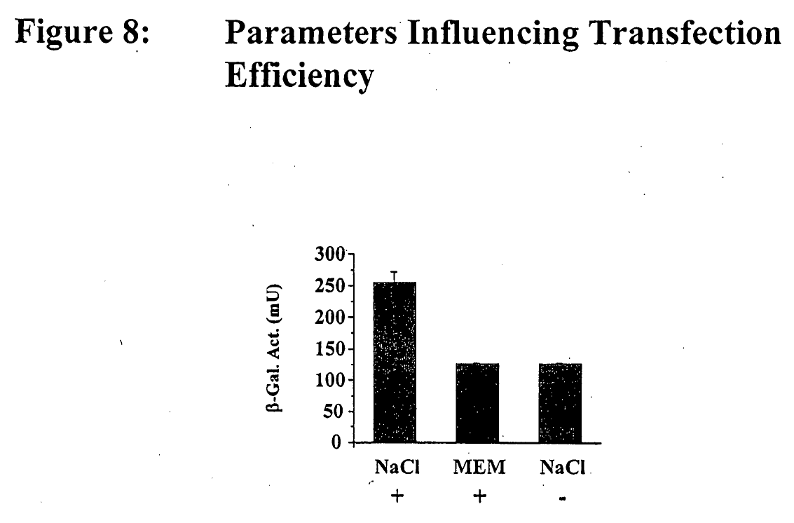

Optimal transfection efficiency was observed when particles were formulated in NaCl as opposed to cell culture medium or glucose (result of transfection in formulation with glucose is not shown). FIG. 8 illustrates the transfection efficiencies obtained. The columns on the left and on the right illustrate transfection efficiencies obtained when the particles were prepared in NaCl; while the centre column designated “MEM” refers to that obtained where the particles were prepared in cell culture medium. The “+” and “−” signs below the columns indicate that the transfections were performed in the presence (+) or in the absence (−) of serum. Taken together, these results suggest that choosing the optimal conditions for formulating PEI-nucleic acid-lipid complexes is advantageous to ensure proper interaction between the polycation, lipid, and nucleic acid and to ensure maximum gene transfer efficiency.

Transfections using PEI-nucleic acid-lipid complexes were also shown to be 2-fold higher when complexes were applied to cells in the presence of serum compared to media without serum (comparison of the two NaCl desigated columns (+) and (−) in FIG. 8). This implies that the presence of serum during transfection does not interfere with formed complexes. The ability to transfect cells in the presence of serum represents a considerable advantage over other transfection methods.

It will be understood by one of ordinary skill in the art that because of this advantage, the compositions of the present invention can be directly applied to cells in culture without requiring prior washing of the cells.

### EXAMPLE 15

### Transfection Efficiency Using Different Formulations and Demonstration of the Suppleness of the Compositions and Methods of the Present Invention

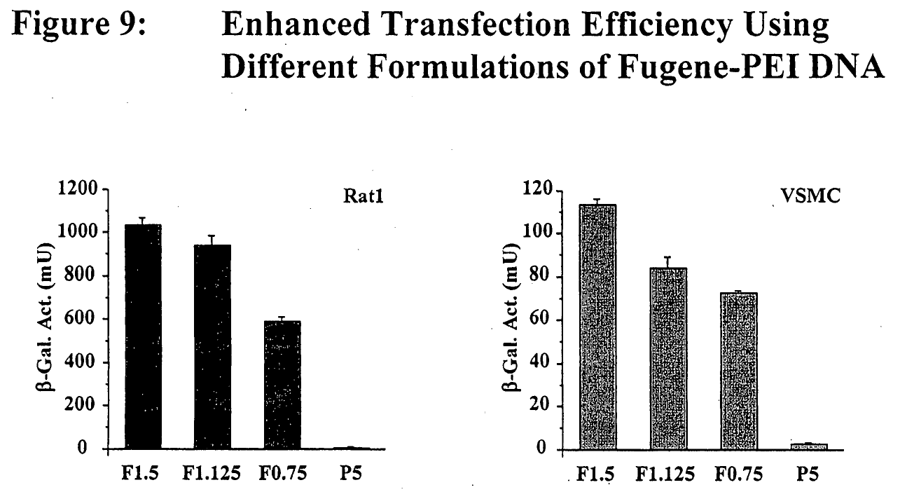

In order to optimize transfection efficiency, different formulations of PEI-nucleic acid-lipid compositions were generated to assess their ability to transfect cells. Various combinations of PEI-FuGENE 6™ were used to complex 1.0 μg of pCMV-LacZ DNA. The N/P ratio was-5 and the FuGENE 6™ amounts varied from 1.50 to 0.75 μl per ug of DNA. Transfection were carried out on Rat1 fibroblast cells (left panel) and VSMC cells (right panel). As seen in FIG. 9, decreasing the amount of lipid present in the complex reduced the transfection efficiency for both cell lines. Interestingly, when half the lipid was added during complex formation, the measured β-galactosidase activity was found to be reduced by less than half. Furthermore, the intermediate quantity of lipid (F1.125) slightly decreased the transfection efficiency for Rat1 fibroblast cells (10% reduction), but had a more pronounced effect on the efficiency when using VSMC (25% reduction). These results indicate that the amount of lipid found in the complex is probably in excess of the amount of lipid required to interact with a given quantity of PEI and DNA present in the complex.

### EXAMPLE 16

### Transfection Efficiency Using Different Formulations and Demonstration of the Suppleness of the Compositions and Methods of the Present Invention

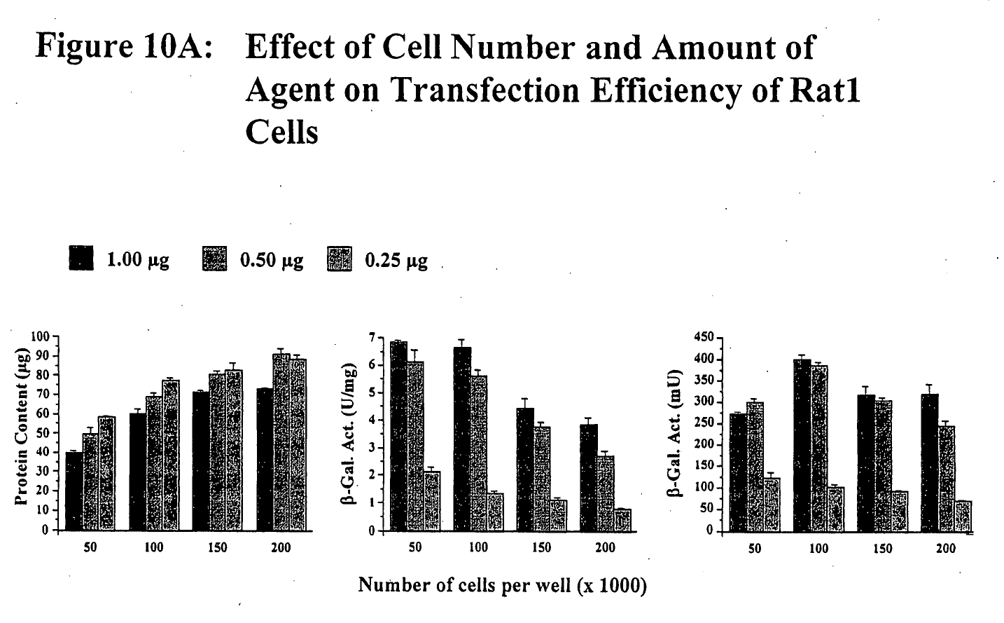

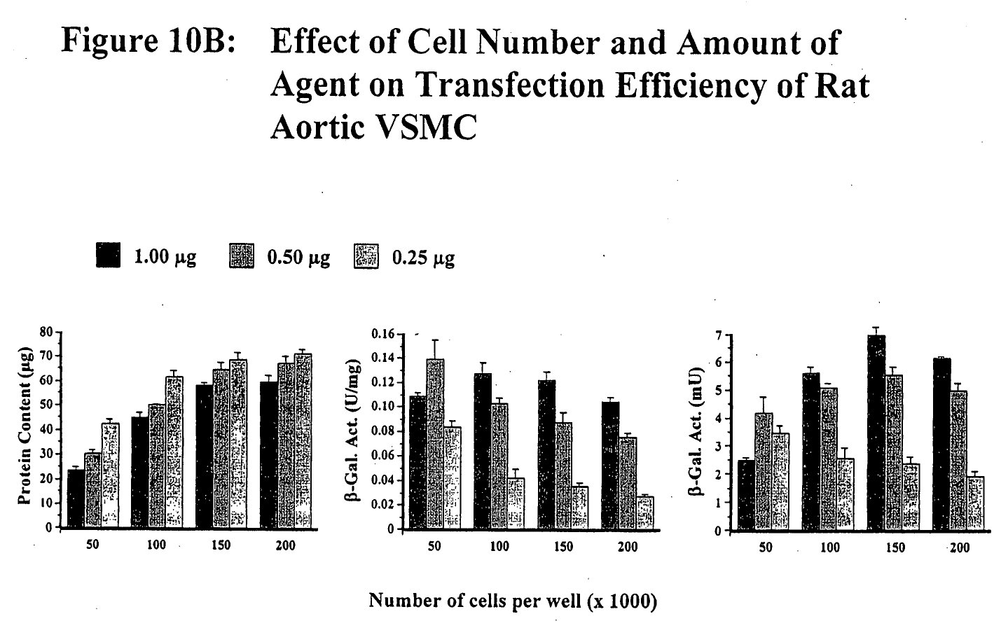

In order to further optimize transfection efficiency, transfections were conducted using different number of cells seeded in each well prior to transfection. The total amount of PEI-nucleic acid-lipid complex represented by the different DNA quantities used was also varied to identify optimal conditions (FIG. 10, A and B). Cells were seeded according to numbers indicated in graph prior to transfection. Transfections were carried out using PEI-FuGENE 6™ to complex plasmid DNA. PEI was used at an N/P ratio of 5 and kept constant. Rat1 fibroblast cells were transfected using 0.75 μl FuGENE 6™ per ug DNA in the complex (A) and VSMCs were transfected with 1.50 μl FuGENE 6™ per ug DNA (B). PEI-FuGENE 6™ DNA complexes were formulated as described in Example 9. Amount of DNA used in each experiment was varied by applying different amounts of total complex generated.

Total β-galactosidase activity as measured in mU reflects total gene product, taking in account U/mg (FIG. 10, A and B, center panels) and protein content (FIG. 10, A and B, left panels) (FIG. 10 A, B; right panels). In the case of Rat1 fibroblast cells, the highest values of total β-galactosidase activity are observed when 100,000 cells were seeded prior to transfection and exposed to complex formed with 1.0 or 0.5 μg of DNA (FIG. 10 A). Hence, among the different tests carried out, these conditions represent the best results, as reflected by the highest activity per mg combined with minimal effect on protein content. Similarly, total β-galactosidase activity was maximal when 150,000 VSMCs were seeded prior to transfection with 1.0 μg DNA (FIG. 10 B).

Furthermore, according to the manufacturer, optimal transfection efficiency using FuGENE 6™ is shown when lipid exceeds DNA (μg) by a factor of three. Use of a ratio of lipid to. DNA Lower than a 3:1 results in decreased transfection efficiency. By using PEI in the transfection complex according to one preferred embodiment of the present invention, the lipid quantity used is reduced by a factor of two so that the final lipid to DNA ratio is 1.5:1.

Taken together, results presented in FIG. 9, 10A and 10B show that the optimal transfection efficiency using the PEI-nucleic acid-lipid complexes can be obtained by varying the composition of the formulation used and the number of cells seeded prior to transfection. Rat1 and VSMCs responded differently to the same formulation of PEI-nucleic acid-lipid probably due to different efficiency with which the complex interacted with each cell line suggesting that some cell lines may be more sensitive to PEI-nucleic acid-lipid complexes and may require less of the composition to achieve optimal transfection efficiency. The PEI-nucleic acid-lipid formulations and methods of the present invention nevertheless display significantly improved transfection efficiencies as compared to those of the prior art. In addition, the present invention exemplifies means to adapt the cationic polymer-nucleic acid-lipid formulation and the use thereof to suit particular needs and particular cells.

Taken together, these results show that the compositions and methods of the present invention are simple and can be adapted by the person of ordinary skill to achieve the desired transfection efficiency without undue experimentation.

### EXAMPLE 17

### Natural, Non-Commercial Lipid Preparations Combined with PEI and Nucleic Acid Enhance Transfection Efficiency

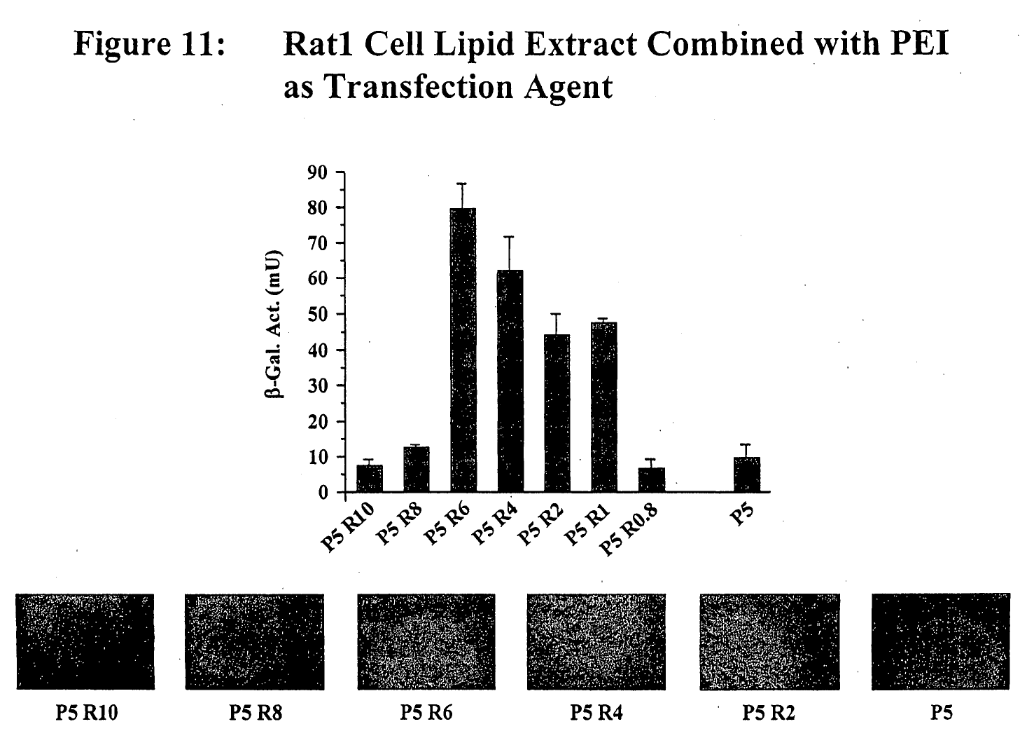

It was verified whether replacing FuGENE 6™ by a natural lipid preparation could also enhance the transfection efficiency of PEI-nucleic acid complexes. Rat1 fibroblast cells lipid extracts preparations were diluted in MEM media and the different dilutions (R0.8 to R10) were combined with PEI (N/P=5) and 1 μg plasmid DNA Cells were lysed 24-48 h post-transfection and the lysate was used to measure protein content and β-galactosidase activity. Cells corresponding to each condition were rinsed in PBS and fixed with glutaraldehyde before addition of X-Gal staining solution as shown in the bottom panels. As may be seen in FIG. 11, as much as an 8-fold increase over transfection efficiency using PEI alone was observed with these natural lipid preparations.

### EXAMPLE 18

### Transfection with Compositions Comprising Synthetic Lipids Combined with PEI and Nucleic Acid

It was verified whether synthetic lipids could be used to replace FuGENE 6™ and enhance the transfection efficiency of PEI-nucleic acid complexes in Rat-1 fibroblast cells. Lipid preparations consisting of lipids 1,2-Dioleoyl-sn-Glycero-3-Phosphoethanolamine (DOPE); 1,2-Dipalmitoleoyl-sn-Glycero-3-Phosphocholine (DPPC); 1,2-Dioleoly-sn-Glycero-3-Phosphocholine (DOPC); 1,2-Diphytanoyl-sn-Glycero-3-Phosphoethanolamine (DPPE); L-α-phosphatidylcholine; 1,2-dioleoyl-3-dimethylammonium-propane; 1,2-dioleoyl-3-trimethylammonium-propane; 1,2-dioleoyl-sn-glycero-3-ethylphosphocholine were diluted in 95% ethanol at a concentration of 2 mg/ml. Lipid preparations were diluted in MEM media and different dilutions were combined with PEI (N/P=10) and 1 μg DNA. Cells were lysed 24-48 h post-transfection and the lysate was used to measure protein content and β-galactosidase activity. L-α-phosphatidylcholine; 1,2-dioleoyl-3-dimethylammonium-propane; 1,2-dioleoyl-3-trimethylammonium-propane and 1,2-dioleoyl-sn-glycero-3-ethylphosphocholine, did not display, alone or in combination with PEI, transfection efficiencies that were better or even equal to that obtained with PEI alone.

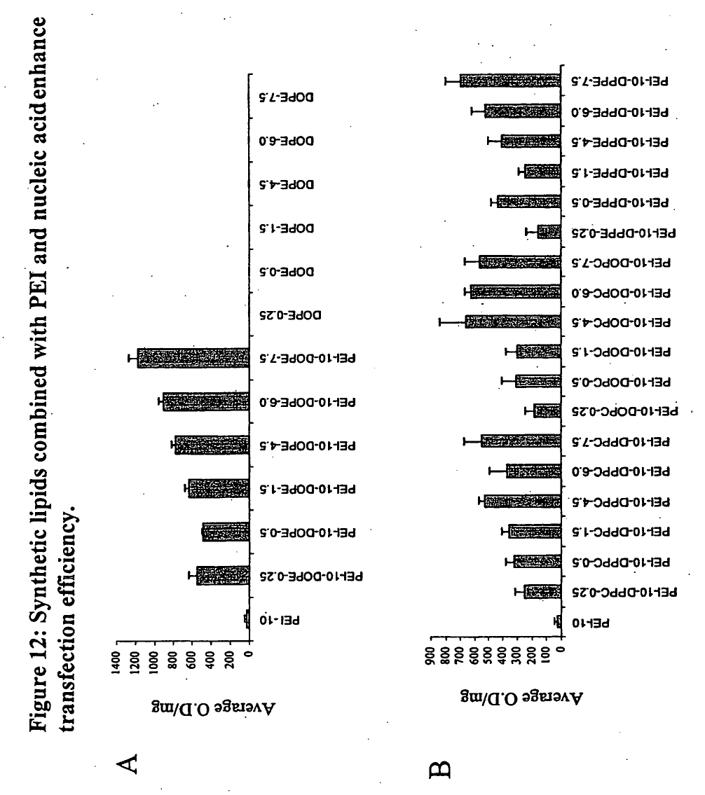

Results of transfections with the other lipids are presented in table 2 below and graphically illustrated in FIG. 12.

TABLE 2

| Agent | AVERAGE O.D./mg | SEM O.D./mg |
|---|---|---|
| PEI-10 | 30.418 | 17.917 |
| PEI-10-DOPE-0.25 | 551.476 | 84.042 |
| PEI-10-DOPE-0.5 | 487.379 | 9.519 |
| PEI-10-DOPE-1.5 | 635.657 | 44.644 |
| PEI-10-DOPE-4.5 | 772.463 | 47.271 |
| PEI-10-DOPE-6.0 | 903.941 | 52.138 |
| PEI-10-DOPE-7.5 | 1180.467 | 96.262 |
| DOPE-0.25 | 0.000 | 0.000 |
| DOPE-0.5 | 0.000 | 0.000 |
| DOPE-1.5 | 0.000 | 0.000 |
| DOPE-4.5 | 0.000 | 0.000 |
| DOPE-6.0 | 0.000 | 0.000 |
| DOPE-7.5 | 0.000 | 0.000 |
| PEI-10-DPPC-0.25 | 256.73 | 57.63 |
| PEI-10-DPPC-0.5 | 323.83 | 53.54 |
| PEI-10-DPPC-1.5 | 360.23 | 43.81 |
| PEI-10-DPPC-4.5 | 529.54 | 39.57 |
| PEI-10-DPPC-6.0 | 368.11 | 128.10 |
| PEI-10-DPPC-7.5 | 552.70 | 121.21 |
| PEI-10-DOPC-0.25 | 186.66 | 60.14 |
| PEI-10-DOPC-0.5 | 310.35 | 91.93 |
| PEI-10-DOPC-1.5 | 302.44 | 72.51 |
| PEI-10-DOPC-4.5 | 662.60 | 173.05 |
| PEI-10-DOPC-6.0 | 624.04 | 42.81 |
| PEI-10-DOPC-7.5 | 561.92 | 103.41 |
| PEI-10-DPPE-0.25 | 160.26 | 76.90 |
| PEI-10-DPPE-0.5 | 432.51 | 45.33 |
| PEI-10-DPPE-1.5 | 245.89 | 45.59 |
| PEI-10-DPPE-4.5 | 404.22 | 97.11 |
| PEI-10-DPPE-6.0 | 518.98 | 97.11 |
| PEI-10-DPPE-7.5 | 695.95 | 99.32 |

1,2-Dioleoyl-sn-Glycero-3-Phosphoethanolamine (DOPE) was shown to enhance transfection efficiency over a wide range of amounts varying from 0.25 to 7.5 μl per ug DNA transfected when combined to PEI (FIG. 12 A and Table 1). Similar amounts of DOPE displayed no β-galactosidase activity when combined to DNA alone. Furthermore, DOPE combined with PEI was shown to enhance transfection efficiency by as much as a 26-fold increase over PEI alone efficiency (FIG. 12 A). 1,2-Dipalmitoleoyl-sn-Glycero-3-Phosphocholine (DPPC), 1,2-Dioleoly-sn-Glycero-3-Phosphocholine (DOPC), and 1,2-Diphytanoyl-sn-Glycero-3-Phosphoethanolamine (DPPE) were also shown to enhance transfection efficiency when combined to PEI as compared to PEI alone (FIG. 12B). These results strongly demonstrate that different lipids allow PEI and DNA to interact more favorably thereby leading to enhanced transfection efficiencies. The synergistic effect of combining PEI to these lipids also indicates that PEI-lipid compositions should not be limited to these exemplified lipids.

### EXAMPLE 19

### Transfection Efficiency Using Different Synthetic Lipid Combinations and Ratios

Various combinations of lipids were prepared to verify if these combinations could enhance the transfection efficiency of PEI-nucleic acid complexes.

All possible combinations of two lipids among 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), and 1,2-Diphytanoyl-sn-Glycero-3-Phosphoethanolamine (DPPE) were prepared as in Example 18. Each two lipid combination was prepared in three different ratios, namely 1:1, 1:3 and 3:1. Total lipid amounts used in all transfections were 4.5 μl/μg DNA.

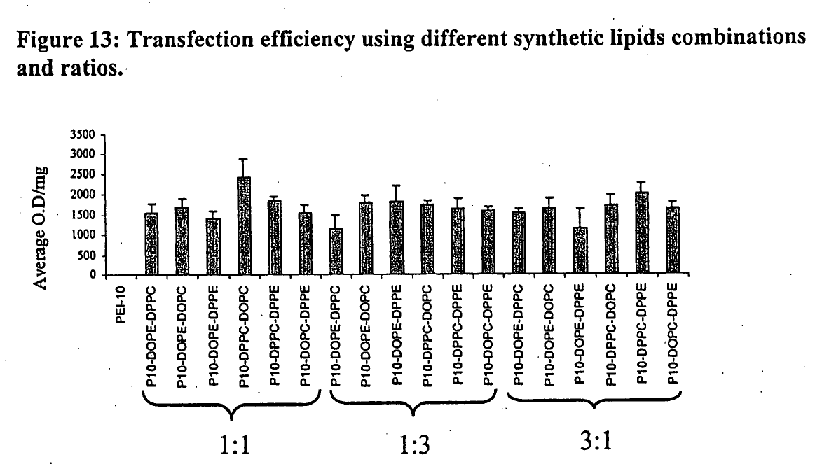

The results of these transfections are presented in FIG. 13, and show that transfection efficiency tested in Rat1 fibroblast cells could also be significantly enhanced over PEI-nucleic acid compositions by using synthetic lipids in different combinations and in different ratios.

Taken together, results presented in Examples 17-19 as well as examples relating to PEI-nucleic acid-FuGENE™ suggest that good transfection efficiency using PEI-nucleic acid-lipid complexes can be obtained with a number of lipids including natural cell lipid extracts, a number of commercial lipid preparations, and DOPE, DPPC, DOPC and DPPE. It is interesting to note that a number of common characteristics may be found in many of these lipids. For instance, natural cell lipid extracts, DOPE, DPPC, DOPC and DPPE are all phosphate containing lipids (cell membranes). Also, DOPE, DPPC, DOPC and DPPE are constituted of acyl chains of 16 or 18 carbons. Cell lipids also contain 16 and 18 carbon lipids. Finally, DOPE and DPPE contain phosphoethanolamine as headgroup; while DOPC and DPPC contain phosphocoline as headgroup. Applicants therefore suggest that although the present invention should not be so limited, lipids containing one or more of these characteristics are useful as lipids for use in the present invention. In accordance with a preferred embodiment of the present invention, the lipid comprised within the cationic polymer-nucleic acid-lipid complex, is a phospholipid. In another preferred embodiment, this phospholipid is constituted of acyl chains of 16 to 18 carbons. In yet another preferred embodiment of the present invention, such phopholipids having acylated chains of 16-18 carbons contain a phosphoethanolamine or phosphocoline head group. In yet an additionally preferred embodiment, the cationic polymer in these complexes is PEI.

It should be understood that the compositions of the present invention are not limited to a single type of lipid or cationic polymer. For certainty, more than one lipid (or polymer) can be used to prepare a cationic polymer-nucleic acid-lipid complex or formulation.

### EXAMPLE 20

### Transfection of Modified Phosphothiurate Fluorescent Oligonucleotides in Rat-1 Cells

Modified nucleic acids such as phosphothiurate fluorescent oligonucleotides provide a useful tool to directly verify uptake of nucleic acid as visualized by fluorescence microscopy. Cells were transfected with 100 nmols of modified phosphotiurate fluorescent oligonucleotide complexed to PEI (N/P=10) and FuGENE 6™ (lipid ratio 1.5:1). 48 hours following transfection, the cells were fixed and assayed visually using fluorescent microscopy. Approximately 80% of Rat-1 cells were fluorescent suggesting that PEI-lipid complexed to modified oligonucleotides represents an efficient method to deliver this type of nucleic acid in cells.

### EXAMPLE 21

### Transfection of Fluorescent RNAi in Rat-1 Cells

Fluoresceine RNAi provides a useful tool to directly verify uptake as visualised by fluorescence microscopy. Rat-1 cells were transfected with 3 μl of 20 μM solution of RNAi complexed to PEI (N/P=10) and FuGENE 6™ (lipid ratio 1.5:1). 48 hours following transfection, the cells were fixed and assayed visually using fluorescent microscopy. A large proportion of cells were fluorescent suggesting that PEI-lipid complexed to RNAi represents an efficient method to deliver this type of nucleic acid in cells. It also demonstrates that nucleic acid other than DNA can successfully be transfected into cells with the compositions and methods of the present invention.

### EXAMPLE 22

### In Vivo Studies

In vivo studies were conducted on mice to verify the toxicity and possible adverse side effects of the transfection reagent. Mice were injected intravenously with PEI-DNA complexes and FuGENE 6™-PEI-DNA complexes. Results show that both complexes, when administered in vivo, did not have any adverse effect on the mice (data not shown), contrary to previous published results showing that mice died within a few minutes following injection of DNA-PEI complexes at low N/P ratios (Goula et al., 1998, Gene Therapy 5:1291-5).

### EXAMPLE 23

### Transfection Efficiency of Mouse Embryonic Fibroblasts

Primary mouse embryonic fibroblasts (MEFs) are considered another type of low transfection generating cells. These cells were transfected with the PEI-DNA lipid complex in order to verify transfection efficiency. Cells were transfected with 1.0 μg of pCMV-LacZ plasmid DNA complexed to PEI (N/P=5) and lipid ratio of 1.5:1. 48 hours following transfection, the cells were fixed and the presence of β-galactosidase was assayed visually by X-Gal staining. Approximately 10% of the MEF cells were shown to be transfected. Taken together, these results show that another cell type known to be refractory to transfection can be transfected with significantly improved efficiency using the compositions and methods of the present invention.

### EXAMPLE 24

### Transfection of Primary Neuronal and Astrocyte Cells

Neuronal cells were isolated from newborn mice and plated prior to transfection procedure. Cells were transfected with β-galactosidase reporter gene plasmid using PEI and FuGENE 6™ as a transfection agent. Transfections were carried out using 1 μg DNA complexed with PEI (N/P=5) and lipid ratio of 3:1. Cells were transfected for 6 hours and β-galactosidase activity was assessed using X-gal staining 36-48 hours following transfection. Staining was observed in both neurons and astrocytes suggesting that the transfection agent successfully entered the cells. Of note, primary neuronal cells and astrocytes are generally recognized as cells which are very refracting to transfection.

Results presented in FIG. 2 to 7 and in above Examples indicate that the same PEI-nucleic acid-lipid formulation can be used to transfect different cell lines that respond differently when transfected with commercial agents. In both Rat1 fibroblasts and VSMCs, the PEI-nucleic acid-lipid formulation increased significantly the transfection efficiencies. Furthermore, results in MEFs, neurons and astrocytes also displayed interesting transfection results. Taken together, these results suggest that the transfection compositions of the invention increase transfection yield of a wide variety of cell lines. Furthermore, these results suggest that the improved transfection yields are based on properties that are intrinsic to the PEI-nucleic acid-lipid formulation and that the degree at which the formulation can successfully transfect a cell line is dependent on the cell line itself.

### EXAMPLE 25

### Statistics

Each experiment described herein was done in triplicate. All values are expressed as means±s.e.m.

Although the present invention has been described hereinabove by way of preferred embodiments thereof, it can be modified without departing from the spirit and nature of the subject invention as defined in the appended claims.

### References

Abdallah et al., 1996, Human Gene Therapy 7:1947-54.

Baker et al., 1997, Gene Therapy 4:773-82.

Bandyopadhyay et al., 1998, Biotechniques 25:282-4, 286-92.

Behr, J. P., 1996, Comptes Rendus des Séances de la Société de Biologie et de Ses Filiales 190:33-8.

Behr et al., 1989, Proceedings of the National Academy of Sciences of the United States of America 86:6982-6.

Boussif et al., 1995, Proceedings of the National Academy of Sciences of the United States of America 92:7297-301.

Boussif et al., 1996, Gene Therapy 3:1074-80.

Dunlap et al., 1997, Nucleic Acids Research 25:3095-101.

Erbacher et al., 1999, Gene Therapy 6:138-45.

Eustice et al., 1991, Biotechniques 11:739-42.

Feigner et al., Human Gene Therapy 8:511-2.

Godbey et al., 1999, Proceedings of the National Academy of Sciences of the United States of America 96:5177-81.

Goula et al., 1998, Gene Therapy 5:1291-5.

Goula et al., 1998, Gene Therapy 5:712-7.

Meunier-Durmort et al., 1997, Gene Therapy 4:808-14.

Mislick et al., 1996, Proceedings of the National Academy of Sciences of the United States of America 93:12349-54.

Ogris et al., 1998, Gene Therapy 5:142-533.

Pollard et al., 1998, Journal of Biological Chemistry 273:7507-11.

Tang et al., 1997, Gene Therapy 4:823-32.

Wagner et al., 1991, Proceedings of the National Academy of Sciences of the United States of America 88:4255-9.

1. A composition for transfecting cells comprising a cationic polymer and a lipid component.

2. The composition as recited in claim 1 wherein said cationic polymer is polyethylenimine.

3. The composition as recited in claim 1, wherein said lipid component comprises a phosphate containing lipid.

4. The composition as recited in claim 1, wherein said lipid component comprises a amino-containing phospholipid.

5. The composition as recited in claim 1, wherein said lipid component comprises a lipid having an acyl chain selected from 16 or 18 carbons.

6. The composition as recited in claim 1, wherein said lipid component comprises a lipid having a headgroup selected from the group consisting of phosphoethanolamine and phosphocholine.

7. The composition as recited in claim 1, wherein the lipid component comprises a lipid selected from the group consisting of a natural lipid preparation, 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), 1,2-Diphytanoyl-sn-Glycero-3-Phosphoethanolamine (DPPE) and any combination thereof.

8. The composition as recited in claim 1, wherein the lipid component comprises 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), 1,2-Diphytanoyl-sn-Glycero-3-Phosphoethanolamine (DPPE) and any combination thereof.

9. The composition as recited in claim 5, wherein the headgroup is phosphoethanolamine.

10. The composition as recited in claim 8, wherein the lipid component comprises DOPE.

11. The composition as recited in claim 5, wherein the headgroup is phosphocholine.

12. The composition as recited in claim 1, wherein the lipid comprises a lipid selected from the group consisting of a natural lipid preparation, 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), and 1,2-Diphytanoyl-sn-Glycero-3-Phosphoethanolamine (DPPE), FuGENE 6™; Effectene™; Lipofectamine™, Lipofectine™ and any combination thereof.

13. The composition as recited in claim 1, further comprising a nucleic acid.

14. The composition of claim 13, wherein said nucleic acid is selected from the group consisting of synthetic, natural or modified DNA, RNA and DNA-RNA hybrids.

15. The composition as recited in claim 13, wherein said nucleic acid is DNA.

16. The composition as recited in claim 14, wherein said DNA has a length of about 10 kb.

17. The composition as recited in claim 1, wherein said lipid is FuGENE 6™.

18. The composition as recited in claim 2, wherein the ratio of polyethylenimine nitrogen/nucleic acid phosphate (N/P) is between about 10 to about 15.

19. The composition as recited in claim 1 for transfecting cells in the presence of serum.

20-23 (cancelled)

24. A method for transfecting cells comprising
a) applying a transfection enhancing amount of the composition of claim 1 to cells to be transfected; and
b) incubating said cells for a time sufficient to enable the transfection thereof.

25. The method of claim 24 wherein said cells are low transfection generating cells.

26. A method for transfecting cells in the presence of serum, comprising incubating said cells with the composition of claim 1 in the presence of serum, for a time sufficient to enable the transfection of said cells.

27. A kit for use in transfecting low transfection generating cells comprising
a) a cationic polymer; and
b) a lipid component.

28. The kit as recited in claim 27, wherein the cationic polymer is polyethyleneimine, and the lipid component comprises a phospholipid.

29. The kit as recited in claim 27, wherein the lipid component comprises a lipid having an acyl chain selected from the group consisting of 16 carbons and 18 carbons.

30. The kit as recited in claim 27, wherein said lipid component comprises a lipid having a headgroup selected from the group consisting of phosphoethanolamine and phosphocholine.

31. The kit as recited in claim 27, wherein the lipid component comprises a lipid selected from the group consisting of natural lipid preparation, 1,2-Dioleoyl-sn-Glycero-3-Phosphoethanolamine (DOPE), 1,2-Dipalmitoleoyl-sn-Glycero-3-Phosphocholine (DPPC), 1,2-Dioleoly-sn-Glycero-3-Phosphocholine (DOPC), 1,2-Diphytanoyl-sn-Glycero-3-Phosphoethanolamine (DPPE) and any combination thereof.

32. The kit as recited in claim 27, wherein the lipid component comprises DOPE.

33. The method as recited in claim 25, wherein said low transfection generating cells are non-dividing cells.

34. The method as recited in claim 25, wherein said low transfection generating cells are primary cells.

35. The method as recited in claim 25, wherein said low transfection generating cells are selected from the group consisting of fibroblasts, vascular smooth muscle cells, neurocytes, astrocytes, mammary epithelial cells, embryonic fibroblasts, hepatocytes, keratinocytes, neurons, oligodendrocytes, embryonic stem cells, HEK-293 (human epithelial kidney), CHO (hamster epithelial ovary), COS (monkey kidney fibroblast), HeLa (human epithelial cervix carcinoma), MCF-7 (human epithelial breast cancer), MEF (mouse embryo fibroblast), SF9 (fall armyworm epithelial ovary), and U-937 (human monocyte).
